(19) United States
(12) Patent Application Publication
(10) Pub. No.: US 2004/0078030 A1
(43) Pub. Date: Apr. 22, 2004

(54) Methods and apparatus for presbyopia treatment using a dual-function laser system

(76) Inventor: J. T. Lin, Oviedo, FL (US)

Correspondence Address: J. T. Lin, 4532 Old Carriage Trail, Oviedo, FL, 32765, US

(21) Appl. No.: 10/270,617

(22) Filed: Oct. 16, 2002

(51) Int. Cl. A61B 18/20

(52) U.S. Cl. 606/5

(57) ABSTRACT

Presbyopia is treated by a method which uses various lasers to remove a portion of the scleral tissue and increase the accommodation of the presbyopic patient's eye By changing the laser power density, fluency or spot size, a single laser device having dual-function of ablation and coagulation is proposed for minimum bleeding. Fiber-bundle coupled to a single fiber is presented to increase the power density of the laser for efficient tissue ablation New mechanisms of lens curvature change and lens anterior shift are proposed for the total accommodation. The preferred laser wavelength ranges from ultraviolet to infrared including (0.15-0.36) microns, (0.9-1.6) microns, (1.8-2.2) microns and (2.8-3.2) microns Both scanning and fiber delivered systems are proposed.

### BACKGROUND OF THE INVENTION

1. Field of the Invention

The present invention relates to apparatus and methods for the treatment of presbyopia using fiber-coupled, dual-function lasers to ablate and coagulate the sclera tissue

2 Prior Art

When a person reaches a certain age (around 45), the eyes start to lose their capability to focus sharply for near vision. Presbyopia is not due to the cornea but comes about as the lens loses its ability to accommodate or focus sharply for near vision as a result of loss of elasticity that is inevitable as people age. The existing vision correction methods are mainly for the treatment of myopia, hyperopia and astigmatism by reshaping the cornea surface Wearing bifocal lens (glasses) has been the major means for the correction of presbyopia, although some surgical methods have been used in clinical trials.

Prior art of Sand's patent (U.S. Pat. No. 5,484,432) uses a thermal laser with spectrum of (1.80-2.55) run to shrink the corneal shape (inside the limbus area). Cold lasers of Lin's prior arts in U.S. Pat. No. 6,258,082 was proposed to ablate scleral tissue (in area outside limbus) for the treatment of presbyopia without changing the shape of the cornea surface In prior art of Rultz (U.S. Pat. No. 5,533,997), multifocal on corneal surface was also proposed to change the curvature of the cornea by ablation the surface layer of the cornea However, Rultz's “presbyopia” correction is fundamentally different from that of the present patent which does not change the corneal curvature and only ablate the scleral tissue outside the limbus area The technique used in the prior art of Bille (U.S. Pat. No. 4,907,586) is specified to conditions of quasi-continuous laser having pulse duration less than 10 picoseconds and focused spot less than 10 micron diameter and the laser is focused into the lens of an eye to prevent presbyopia He also proposed to use laser to create a cavity within the corneal stroma to change its visco-elastic properties.

To treat presbyopic patients, or the reversal of presbyopia, using the concept of expanding the sclera by mechanical devices has been proposed by Schachar in U.S. Pat. Nos. 5,489,299, 5,722,952, 5,465,737 and 5,354,331 These mechanical approaches have the drawbacks of complexity and are time consuming, costly and have potential side effects. To treat presbyopia, the Schachar U.S. Pat. Nos. 5,529,076 and 5,722,952 propose the use of heat or radiation on the corneal epithelium to arrest the growth of the crystalline lens and also propose the use of lasers to ablate portions of the thickness of the sclera. However, these prior arts do not present any details or practical methods or laser parameters for the presbyopic corrections No clinical studies have been practiced to show the effectiveness of the proposed concepts by Schachar and many of his proposed lasers are thermal lasers which will cause thermal burning of the cornea, rather than tissue ablation. Schachar's methods also require the weakening of the sclera and increase the lens diameter by expansion

Another prior art proposed by Spencer Thornton (Chapter 4, “Surgery for hyperopia and presbyopia”, edot3d by Neal Sher (Williams & Wilkins, MD, 1997) is to use a diamond knife to incise radial cuts around the limbus areas. It requires a deep (90%-98%) cut of the sclera tissue in order to obtain accommodation of the lens This method, however, involves a lot of bleeding and is difficult to control the depth of the cut which requires extensive surgeon's skill

Lin's prior arts in U.S. Pat. No. 6,258,082 proposed the use ablative lasers to ablate scleral tissue and cause the increase of the elasticity of the sclera-ciliary-zonules complex for improved accommodation of presbyopic patients One of the major drawbacks of surgical method for the treatment of presbyopia is the inevitable bleeding which occurs when cutting the conjunctival or scleral tissue Conventionally, this bleeding requires electrode device such as bipolar to stop Another prior art of Lin in U.S. Pat. No. 6,263,879, proposed a “dual-laser” system using an ablative laser and a coagulative laser for the treatment of presbyopia This prior art, however requires a scanning device and combining of two different lasers to achieve ablation and coagulation effects These two lasers are also required to interact with the tissue in a totally different nature, one “cold” and one “thermal”, which are difficult and costly to make for practical applications

Regarding the mechanisms cause the increase of accommodation of an presbyopic eye, prior arts of Schachar proposed ciliary-body “expansion” (U.S. Pat. Nos. 5,465,737 and 5,354,331) which was in contrary to the recent measurements of ciliary-body “contraction” for patient to see near. More recently, Lin (PCT/US/01/24618) proposed a new mechanism of sub-conjunctiva filling to explain the increase of accommodation and minimum regression after surgery All prior arts, however, are proposing the lens curvature change is the “only” factor for accommodation In the present patent, we propose additional mechanisms to explain the total accommodation.

One objective of the present invention is to provide an apparatus and method to obviate these drawbacks in the above-described prior arts

It is yet another objective of the present invention to use one “single” laser unit to achieve dual function of ablation and coagulation when soft tissue is ablated such that presbyopia treatment can be conducted with minimum bleeding and procedure can be done faster without the use of bipolar device

It is yet another objective of the present invention to identify various means of switching from ablative to coagulative mode for the same laser by the concept of soft tissue “ablation threshold”, where only one laser is needed versus that 2 lasers are required in prior arts

It is yet another objective of the present invention to identify new mechanisms for the increase of accommodation of the presbyopic eye including the anterior movement of the lens

It is yet another objective of the present invention to propose a novel method which combines fiber bundles from diode lasers and re-focus to a smaller single fiber to increase the power density, which otherwise can not be achieved by the current diode laser technology

### SUMMARY OF THE INVENTION

The preferred embodiments of the present surgical laser consists of a combination of an ablative-type laser and a delivery unit The ablative-type laser preferred to have a wavelength range of (015 to 035) um, or (095-16) um, or (185-32) um or (4-10) um and should be operated in a pulsed mode such that the thermal damage of the ablated tissue is minimized.

It is yet another preferred embodiment of the present surgical system to provide means of switching from ablative-mode to coagulative-mode by controlling laser parameters such as power, energy, fluency and beam spot size

It is yet another embodiment of the present surgical laser to provide an integration system in which the dual-mode laser energy may be delivered by a scanner, an articulated arm or a fiber-coupled device

It is yet another embodiment of the present surgical method that the total accommodation of the presbyopic eye shall include both lens curvature change and the anterior movement of the lens.

It is yet another embodiment of the present surgical method that a novel method which combines fiber bundles from diode lasers and re-focused to a smaller single fiber to increase the power density

It is yet another embodiment of the present surgical laser to provide an integration system in which the sclera ablation leads to the increase of the accommodation of the ciliary muscle for the treatment of presbyopia and for the prevention of open angle glaucoma.

Further preferred embodiments of the present surgical laser will become apparent from the description of the invention which follows

### BRIEF DESCRIPTION OF THE DRAWINGS

FIG. 1 shows a two-component model for accommodation, where the image may be shifted for near vision by lens anterior shift (A) or by curvature change of the lens (B).

FIG. 2 shows the threshold power density P0 which defines a laser to be in coagulation mode or ablation mode.

FIG. 3 is a block diagram of an integrated laser system consisting of a basic laser, a mode-controller and coupled to a fiber and a hand piece

FIG. 4 is a block diagram of the hand piece in which a diode laser bundle is coupled to a single fiber by a lens combination.

### DETAILED DESCRIPTION OF THE INVENTION AND THE PREFERRED EMBODIMENTS

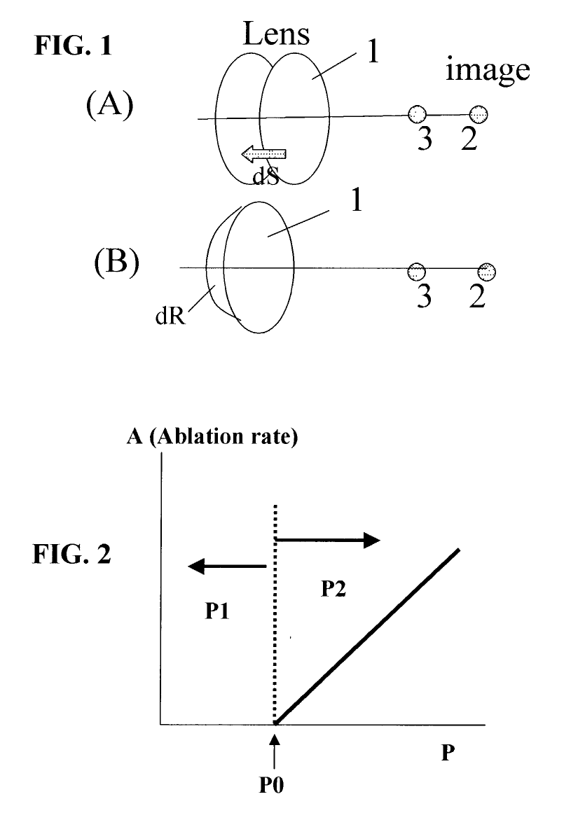

First we shall present the mechanism for accommodation. We define the total accommodation (TA) given by two components the lens curvature change (dR) and the lens anterior shift (dS) As shown in FIG. 1, the lens 1 will have image shifted from position 2 to 3 to see near for presbyopia patients by either the change of the lens curvature (dR) or its shifting (dS) Our calculations (J. T. Lin, unpublished) showed that depending on the initial lens curvature and its anterior chamber depth, the TA is about (03 to 20) diopters for dR change from 105 mm to (1015 to 832) mm and about 0.97 diopters for each 10 mm shift of dS. Therefore for “old lenses” (say age of 50 and up) with rigid lens capsule (or small dR), the main contribution for accommodation is from the anterior shift (dS), whereas for “young lens” (say age of 40-49), the lens curvature shall be the dominate components In our clinical results using an infrared laser, we found about (15 to 30) diopter of accommodation after the surgery. And some cases, there was not effects at all, which we believe is due to the fact that the presbyopic eye was too rigid to be fixed, either by dR or by dS The above proposed concept is in dynamical mode such that the lens can move forward (to the cornea) to see near and move backword when the eye needs to see far. This new concept of two component dynamical model as described above was not proposed earlier

Another new concept of the present patent is presented for a “single” laser to perform both ablation and coagulation as follows As shown in FIG. 2, by the physics of “laser-tissue” interactions, it was known that a laser will behave as a thermal laser when its energy (or power density) is below the so called “ablation threshold” level, the P0 shown in FIG. 2 Depending on types of lasers (wavelength and operation modes), this threshold P0 value may range from (50-500) W/cm sup 2 for laser power density or (1-10) mJ/cm sup.2 for laser fluency. A laser may be controlled by various means such that it can be switched from an “ablation mode” (AM) to a coagulation mode (CM). The preferred control means shall include (for a laser switches from CM to AM): (1) laser energy\pulse control (from low to high), (2) laser fluency control, by changing laser beam spot size (from small to big spot), where the fluency is defined by energy/spot area); (3) laser operation model control (from continuous mode to a pulsed mode, such that the peak power density increases); and (4) switching laser wavelength from a thermal-mode (having weaker tissue absorption, such as spectra range of 980-1300 nm) to an ablation mode (having strong absorption such as UV-laser of 02-03 um, IR lasers of 145, 2.8-3.2 um). In method (4) frequency conversion nonlinear crystals shall be required for this purpose

One preferred example is an Nd:YAG lasers with UV (355 nm or 266, or 215 nm) outputs and can be switched from low to high power mode, or from continuous-wave (CW) to Q-switched model Second preferred example is to use a diode laser at about 09 um or (14-1.6) um operated at CW mode but spot size may be changed from large (0 8-1 5) mm to small (0.2-0.7) mm, or laser power can be switched from low (0.2-0.5) W to high (2.0-10.0 W). The third preferred example is to use a mid IR laser (2 8-3 2) um operated at free running (about few hundreds of microseconds pulse duration) or CW mode and can be switched for its power level from about (0 1-0 2) W to about (0 3-5 W) or switching its spot size form (0 8-1 5) mm to (0 2-0 7) mm. We shall note that the spot size change (reduced) of 30% produce a power density (or fluency) of 69% more which allows us to control the laser mode from CM to AM In addition, the peak power may increase a factor of 100 by switching from a long pulse (say 1,000 usec) to a short pulse (say 100 usec) mode We have tested an Er YAG laser (at 2 94 um, run at about 200 usec) and a diode laser at about 1.5 um (CW mode) at low power and high power levels by the proposed means and confirmed the control/switch of coagulation/thermal mode (CM) and ablation model (AM) on animal eyes The CM showed some kind of thermally burned “white” spot whereas the AM showed no thermal damage/color with “sharp” ablating edges The threshold energy/pulse (for spot of 0 6 mm) was about 10 mJ in Er YAG laser and threshold power was about 0.3 W (spot of about 0.1 mm) in diode laser at 1.5 um.

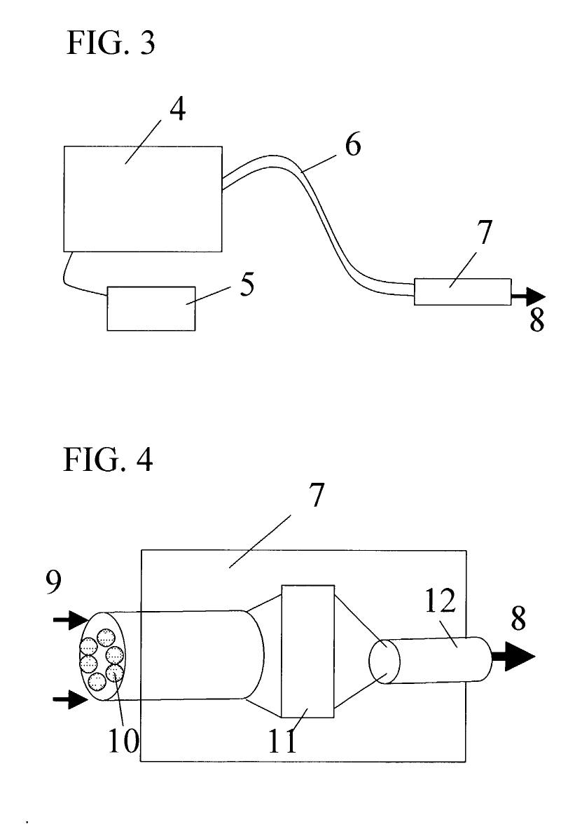

FIG. 3 of the drawings is a schematic of one of the preferred embodiments which is a surgical system having the basic laser 4 controlled by a microprocessor 5 and coupled to a fiber 6 having a hand piece 7 with output laser 8. The microprocessor controls the laser power density or fluency (at a given spot size) such that the laser may be in an ablation mode (AM) or coagulation mode (CM) The output beam 8 may have a typical spot size of (0 3-0 6) mm in AM and larger spot of (0 8-1 5) in CM, or both have the same spot size but have different power/fluency level The preferred basic laser 8 shall have a predetermined wavelength of (0 19-0 36) um, (0 96-0 98) um, (1.45-1.6) um, (1.85-2 2) um or (2 8-3 2) um In these selected laser spectra, the soft tissue shall have certain absorption of the laser power via either water or proteins and therefore we may control the interaction modes of CM or AM We note that we have excluded lasers with spectra range of visible (400-700) um and near infrared (1.0-1 4) um which do not have enough absorption coefficients to perform an ablation mode, although they may cause thermal effects to the tissues For examples, our preferred lasers include a diode laser at about 980 nm has higher absorption than that of 1064 nm (Nd:YAG laser), 1.45 um laser has higher absorption than that of 1.3 or 1 4 um lasers, and lasers at (2.8-32) um have the strongest absorption peaks of water and soft tissues. Another preferred embodiment is to deliver the laser energy to the eye by an articulated arm or scanning device using reflecting mirrors or motorized gavometer to the predetermined areas of the eye, outside the limbus. Typical sclera ablation patterns shall include radial lines or dots with a depth of about (400-600) um.

The preferred embodiments of FIG. 3 shall require the ablative laser to meet one of the peaks of tissue absorption spectra such as 0 98, 1 45, 2 1, 2.94 and 6.0 microns Therefore, the preferred embodiment of the basic laser 4 shall include solid state lasers of Er YAG, Er.YSGG, Ho YAG, optical parametric oscillation (OPO) laser at (2.6-3 2) microns; a gas laser with a wavelength of (2.6-3.2) microns, an excimer laser of ArF at 193 nm; a XeCl excimer laser at 308 nm, a frequency-shifted solid state laser at (0 15-3 2) microns, the harmonic generation of Nd:YAG or Nd:YAL or Ti.sapphire laser at wavelength of about (190-220) nm; a CO laser at about 6 0 microns and a carbon dioxide laser at 10.6 microns; a diode laser at (0 8-2 1) microns, or any other gas or solid state lasers including flash-lamp and diode-laser pumped, at (0 5-10 6) microns spectra range

In FIG. 4, one preferred embodiment of the hand piece 7 is shown The purpose of this device is to convert low-power diode laser fiber-bundle into a single-fiber with high power output. Fiber bundle 10 are coupled into a single fiber 12 by a lens combination 11, whereby the input total power given by N×P1, where P1 is the power of individual bundle and N is the number of bundles The output power 8 from the single fiber 12 is then given by P2=N×P1. For a given fiber dimensions of R1 (input end) and R2 (output end), the power density of the output end (F2) becomes F2=F1×(R1/R2) sup. 2 which higher than that of the input end F1 For R1=1 5 mm and R2=0.5 mm, we may obtain F2 to be nine (9) times of F1. One preferred embodiment of the present invention is to couple an input end consisting of 37 fibers bundle with a power of about 0 4 W each diode at a wavelength of 1 45 um, with each fiber size of 120 um, or F1=354 W/cm sup 2, we are able to obtain an output power density of F2=9×354=3,186 W/cm sup 2 for R1=1 5 mm and R2=0 5 mm. Given the current diode laser technology, it is very costy or difficult to make a KW power density diode from a single fiber at this wavelength of 1 45 um. However by using the above described device, we are able to convert low power density fiber-bundle to a high power density output from a single fiber. In addition, this 1 45 um diode laser (matching one of the water absorption peaks) can be used to effectively ablate soft tissues such as the sclera tissue of an eye when operated at high power, whereas it can also be used to coagulate the tissue when a low power mode is used Without coupling the fiber-bundle to a single fiber with higher power density, the 0 4 W fiber may not be enough to ablate the tissue Similarly other diode lasers with preferred wavelength of (0.96-0.98) um and (1 8-2 2) um may be used in the above described device.

One preferred embodiment of the present surgical laser is to remove a portion of the sclera tissue outside the limbus such that accommodation of a presbyopic eye increases to see near by the mechanisms presented earlier. In addition, by switching the laser mode from an ablation mode (AM) to a coagulation mode (CM), we are able to use one single laser to conduct sclera ablation with minimum bleeding. This dual-function laser system is important strongly desired in the presbyopia treatment which involves with cutting of conjunctiva and sclera tissue and bleeding in inevitable Another preferred embodiment of the present surgical laser is to the prevent or treat glaucoma by reduction of the intra-ocular pressure after a portion of sclera tissue is removed

The invention having now been fully described, it should be understood that it may be embodied in other specific forms or variations without departing from the spirit or essential characteristics of the present invention. Accordingly, the embodiments described herein are to be considered to be illustrative and not restrictive

I claim:

1. An ophthalmic surgery method for treating presbyopic patient by removing a portion of the scleral tissue of an eye by a laser beam having a dual-function of ablation and coagulation with the soft tissue of the eye, whereby the accommodation of the presbyopic eye increases to see near

2. An ophthalmic surgery method for treating presbyopic patient in accordance with claim 1 in which said accommodation of the presbyopic eye increases via the change of the lens curvature

3. An ophthalmic surgery method for treating presbyopic patient in accordance with claim 1 in which said accommodation of the presbyopic eye increases via the anterior movement of the lens.

4. An ophthalmic surgery method for treating presbyopic patient in accordance with claim 1 in which said laser beam is a ultraviolet laser having a predetermined wavelength of about (0 15-0.36) microns

5. An ophthalmic surgery method for treating presbyopic patient in accordance with claim 1 in which said laser beam is an infrared laser having a predetermined wavelength of about (0 95-10 6) microns

6. The method of claim 8 wherein said laser beam includes is a solid-state YAG-based laser frequency shifted to about (190-360) nm.

7. The method of claim 4 wherein said laser beam includes excimer lasers at wavelength of (193-308) nm.

8. The method of claim 4 wherein said laser beam is a solid state Er YAG laser at 2 94 microns

9. The method of claim 5 wherein said laser beam is an infrared semiconductor diode laser with wavelength of about (0 96-0 98) um, (1 45-1 60) um or (1.85-2.20) um

10. An ophthalmic surgery method for treating presbyopic patient in accordance with claim 1 in which said laser beam is delivered to the predetermined area outside the limbus of the eye by an optical fiber connected to a hand piece

11. An ophthalmic surgery method for treating presbyopic patient in accordance with claim 1 in which said laser beam is delivered to the predetermined area outside the limbus of the eye by an articulated arm

12. An ophthalmic surgery method for treating presbyopic patient in accordance with claim 1 in which said laser beam is delivered to the predetermined area outside the limbus of the eye by a scanning device

13. An ophthalmic surgery method for treating presbyopic patient by removing a portion of the scleral tissue of an eye in accordance with claim 1 in which the sclera ablation has a depth of about (400-600) microns.

14. An ophthalmic surgery method for treating presbyopic patient in accordance with claim 1 in which said dual-function of ablation and coagulation is controlled by changing the power density, fluency or spot size of the said laser beam.

15. An ophthalmic surgery method for treating presbyopic patient in accordance with claim 1 in which the power density of said laser beam is increased by a device consisting of a hand piece, a fiber-bundle coupled to a single fiber by a lens combination
